(19) United States
(12) Patent Application Publication
(10) Pub. No.: US 2011/0118563 A1
(43) Pub. Date: May 19, 2011

(54) Portable multi-Parameter physiological monitoring and recording device

(75) Inventors: Chao-Fa Lee, Taipei (TW); Shih-Jung Chang, Taipei (TW); Cheng-Hsing Kuo, Taipei (TW)

(73) Assignee: Tatung Company, Taipei (TW)

(21) Appl. No.: 12/654,915

(22) Filed: Jan. 8, 2010

(30) Foreign Application Priority Data
Nov. 13, 2009 (TW) 098138549

(51) Int. Cl. A61B 5/00

(52) U.S. Cl. 600/301

(57) ABSTRACT

A portable multi-parameter physiological monitoring and recording device includes multiple sensors, multiple detection channel units, a multiplexer, a clock generator, a controller, an analog switch, a recording unit and a storage unit. The sensors are attached on the body of a person for sensing physiological parameter signals. The detection channel units amplify the received physiological parameter signals. The multiplexer captures the amplified physiological parameter signals. The clock generator generates clock signals in the audio-frequency range. The controller controls the multiplexer to capture the amplified physiological parameter signals based on the clock signals generated by the clock generator. The analog switch outputs the amplified physiological parameter signals as an audio signal. The recording unit records the audio signals as an audio file. The storage unit stores the audio file.

### BACKGROUND OF THE INVENTION

1. Field of the Invention

The present invention relates to the technical field of biomedical signal measurement and, more particularly, to a portable multi-parameter physiological monitoring and recording device for monitoring a plurality of physiological signals. The device transforms these physiological signals into a plurality of electrical signals and outputs an audio signal.

2. Description of Related Art

As the scientific and technological progress, nowadays various biomedical devices for measuring physiological signals, such as the high-precision equipments in the hospitals and the home-care devices used at home, are provided to collect the measured analog physiological signals. All these analog signals are converted into digital signals for data storage in the computer system. Therefore, the computer system can do the subsequent analysis, comparison or determination for the measured results accurately. But most people who need to measure or examine these physiological signals in daily life or for a long term period is not good at using complexity computer, it is difficult to use the current biomedical devices.

Therefore, it is desirable to provide a portable multi-parameter physiological monitoring and recording device to mitigate and/or obviate the aforementioned problems.

### SUMMARY OF THE INVENTION

The object of the present invention is to provide a portable multi-parameter physiological monitoring and recording device by attaching multiple sensors on the body of a person. Under test for measuring multiple physiological signals, and the measuring results are outputted as audio signals and can be recorded as an audio file.

To achieve the object, the present invention provides a portable multi-parameter physiological monitoring and recording device, which is used for measuring multiple physiological signals from a person and outputting the results as an audio file. The portable multi-parameter physiological monitoring and recording device includes: multiple sensors, which are attached on specified locations on the body of a person for sensing the corresponding physiological signals respectively; multiple detection channel units, which are respectively connected to the corresponding sensors to amplify the received physiological signals; a multiplexer, which is connected to the detection channel units for capturing the amplified physiological signals; a clock generator, which is used for generating clock signals in the audio-frequency range; a controller, which is connected to the multiplexer and the clock generator, and is used for controlling the multiplexer to capture the amplified physiological signals based on the clock signals generated by the clock generator; an analog switch, which is connected to the detection channel units and the multiplexer, and is used for outputting the amplified physiological parameter signals as an audio signal; a recording unit, which is connected to the analog switch and the controller, and is used for recording the audio signals as an audio file under control by the controller; and a storage unit, which is connected to the recording unit for storing the audio file.

### BRIEF DESCRIPTION OF THE DRAWINGS

FIG. 1 is a schematic diagram of the portable multi-parameter physiological monitoring and recording device according to the present invention;

FIG. 2 is a schematic diagram of audio signal output from the analog switch of the portable multi-parameter physiological monitoring. A recording device could be in accordance with one preferred embodiment of the present invention;

FIG. 3 is a circuit diagram of the portable multi-parameter physiological monitoring and recording device in accordance with one preferred embodiment of the present invention;

FIG. 4a is a circuit diagram of the detection channel unit of room temperature of the portable multi-parameter physiological monitoring and recording device in accordance with one preferred embodiment of the present invention;

FIG. 4b is a circuit diagram of the detection channel unit of body temperature of the portable multi-parameter physiological monitoring and recording device in accordance with one preferred embodiment of the present invention;

FIG. 4c is a circuit diagram of the detection channel unit of XYZ three-axis acceleration of the portable multi-parameter physiological monitoring and recording device in accordance with one preferred embodiment of the present invention;

FIG. 4d is a circuit diagram of the detection channel unit of EKG of the portable multi-parameter physiological monitoring and recording device in accordance with one preferred embodiment of the present invention; and

FIG. 4e is a circuit diagram of the detection channel unit of breathing of the portable multi-parameter physiological monitoring and recording device in accordance with one preferred embodiment of the present invention.

### DETAILED DESCRIPTION OF THE PREFERRED EMBODIMENT

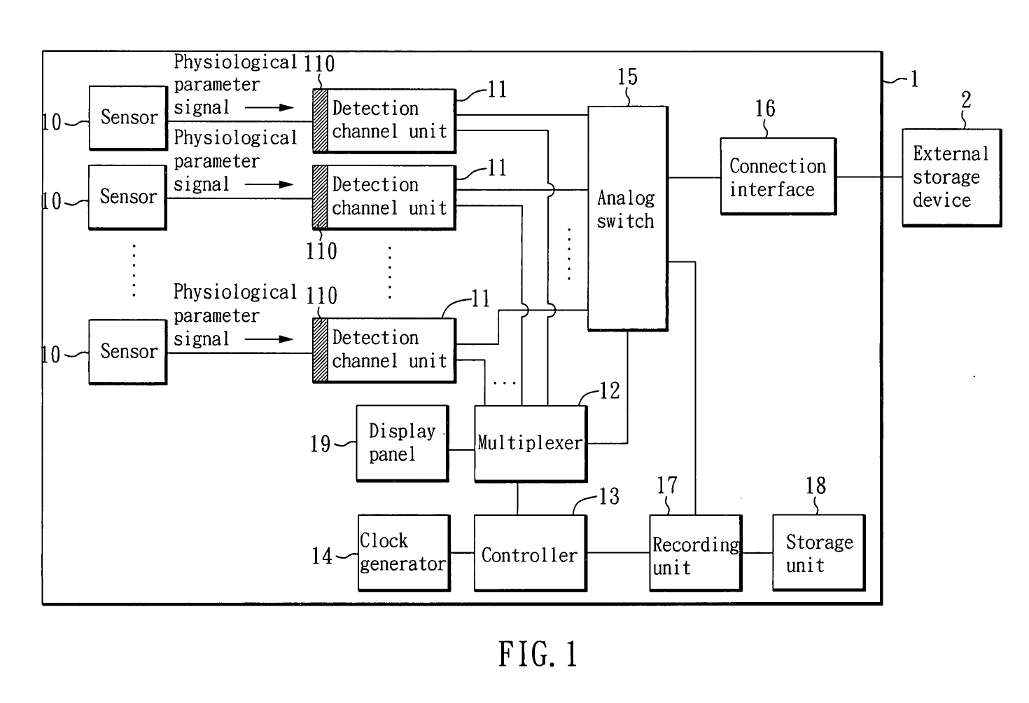

Please refer to FIG. 1. FIG. 1 is a schematic diagram of the portable multi-parameter physiological monitoring and recording device according to the present invention. The portable multi-parameter physiological monitoring and recording device 1 comprises multiple sensors 10, multiple detection channel units 11, a multiplexer 12, a controller 13, a clock generator 14, an analog switch 15, a recording unit 17, a storage unit 18 and a connection interface 16. The sensors 10 are attached on specified locations of the body of a person for sensing the corresponding physiological signals, such Electrocardiography (EKG) signals, breathing signals, body impedance signals, room temperature signals, body temperature signals and XYZ three-axis acceleration signals, respectively. Each of the detection channel units 11 includes an isolation layer 111. The detection channel units 11 are respectively, connected to the corresponding sensors to amplify the received physiological signals, wherein the isolation layers 111 are provided for separating the skin of the person and the circuits of the physiological monitoring and recording device to prevent leakage current or noise from harming the human body. The multiplexer 12 is connected to the detection channel units for capturing the physiological signals amplified by the detection channel units 11. The controller 13 is connected to the multiplexer 12 and the clock generator 14, wherein the clock generator 14 is used for generating clock signals in the audio-frequency range, and the controller 13 is provided for controlling the multiplexer 12 to capture the amplified physiological parameter signals based on the clock signals generated by the clock generator 14. The analog switch 15 is connected to the detection channel units 11, the multiplexer 12 and the connection interface 16 for outputting the physiological parameter signals amplified by the multiplexer 12 as audio signals, wherein the frequency of the audio signals is between 200 Hz and 20 KHz. The recording unit 17 is connected to the analog switch 15 and the controller 13 for recording the audio signals as an audio file under the control of the controller 13. The storage unit 18 is connected to the recording unit 17 for storing the audio file.

When the physiological monitoring and recording device 1 is used for measuring the physiological signals, first, all sensors 10 are attached on specified locations on the body skin of a person. Then, the detection channel units 11 respectively amplify the received physiological signals sensed by the corresponding sensors 10, and the controller 13 controls the multiplexer 12 to capture the amplified physiological signals based on the clock signals generated by the clock generator 14. Finally, the analog switch 15 outputs an audio signal for recording as an audio file by the recording unit 17 and storing the same in the storage unit 18, or the analog switch 15 outputs an audio signal through the connection interface 16.

The physiological monitoring and recording device 1 preferably includes a display panel 19, which is connected to the controller 13 for displaying the amplified physiological parameter signals captured by the multiplexer 12.

The physiological monitoring and recording device 1 is connected to an external storage device 2 through cables or wireless transmission, such as audio cables or Bluetooth wireless transmission. The external storage device 2, which is preferably a computer device, a recording device or a personal digital assistant, is provided for storing the audio signal which is outputted from the connection interface 16 as an audio file. The external storage device 2 is preferably provided to process the received audio signal to proceed analysis or determination instantly, for example, the external storage device 2 issues warning for informing users when the physiological signals are abnormal.

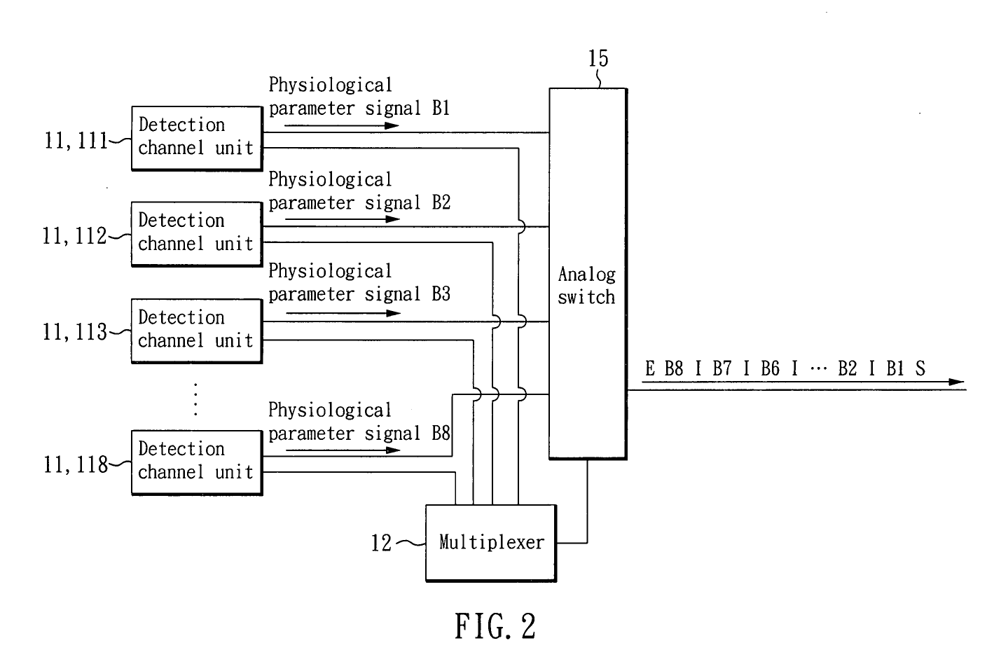

Please refer to FIG. 2. FIG. 2 is a schematic diagram of signal output mode of the analog switch of the portable multi-parameter physiological monitoring and recording device in accordance with one preferred embodiment of the present invention. The analog switch 15 is provided with a first reference voltage value, a second reference voltage value and a third reference voltage value, which preferably are VCC, VSS and GND respectively, for constituting a start signal, an interval signal and a terminate signal. For example, sequential output of VCC, VSS, GND, VSS, VCC, and GND is used as the start signal, output of GND is used as the interval signal, and output of GND, and GND is used as the terminate signal.

As shown in FIG. 2, the detection channel units 111-118 output the physiological parameter signals B1-B8 respectively. The analog switch 15 is used for outputting the amplified physiological parameter signals captured by the multiplexer 12 in an output mode of sequentially outputting the start signal S, the physiological parameter signal B1, the interval signal I, the physiological parameter signal B2, the interval signal I, the physiological parameter signal B3, the interval signal I, . . . the physiological parameter signal B8, the terminate signal E.

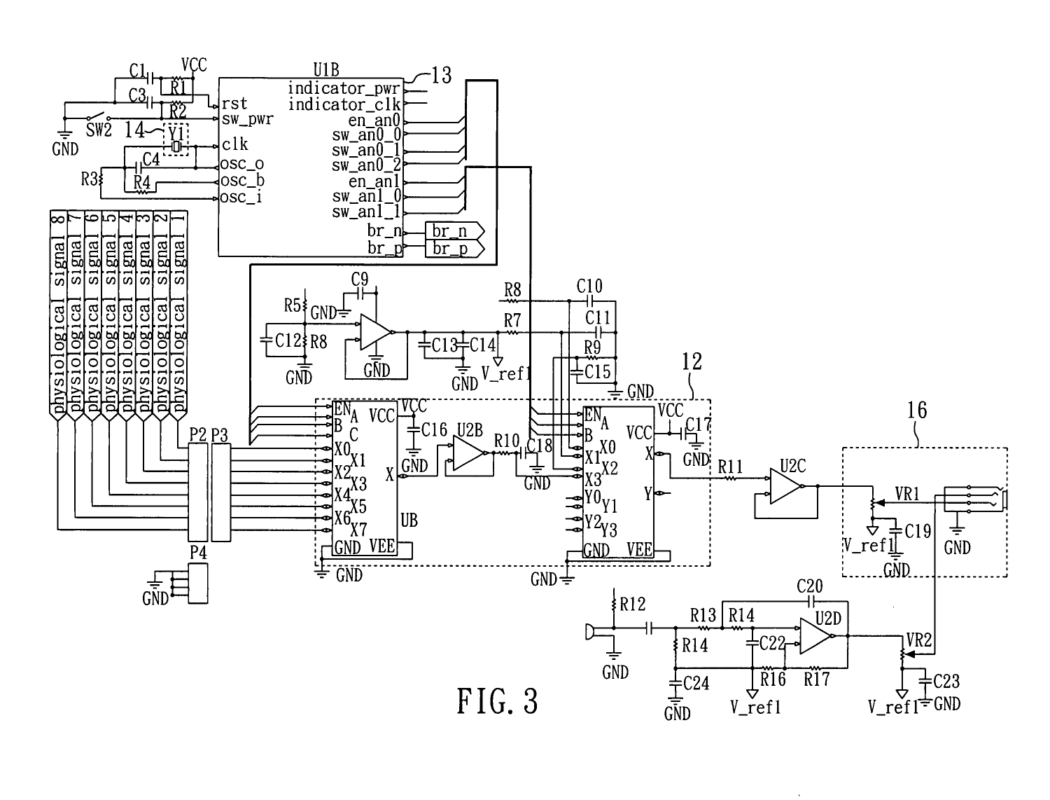

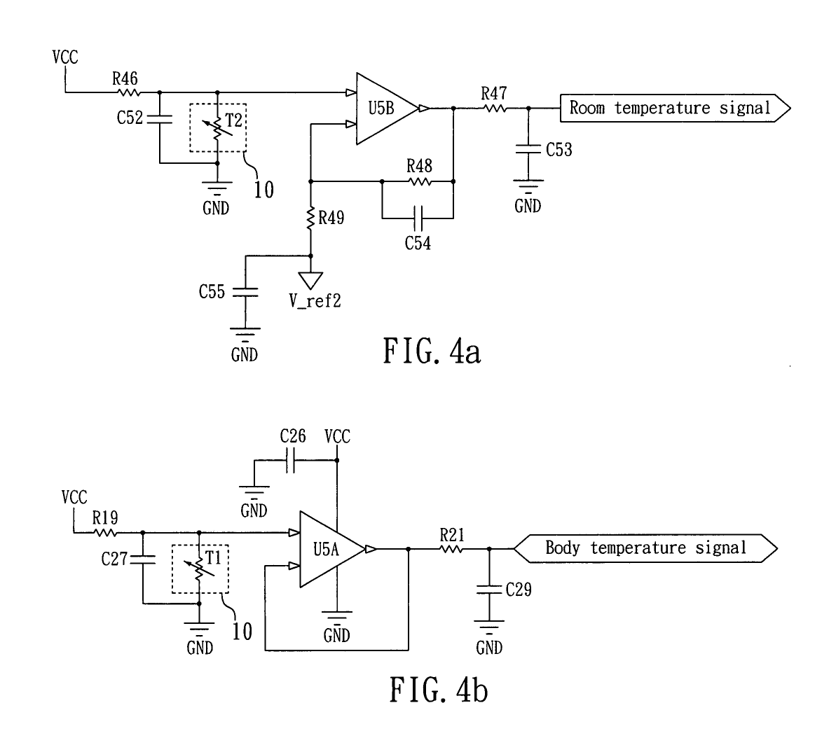

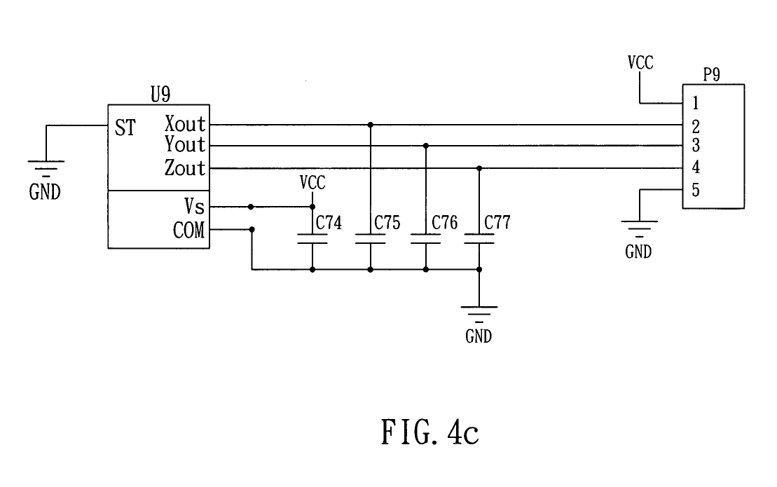

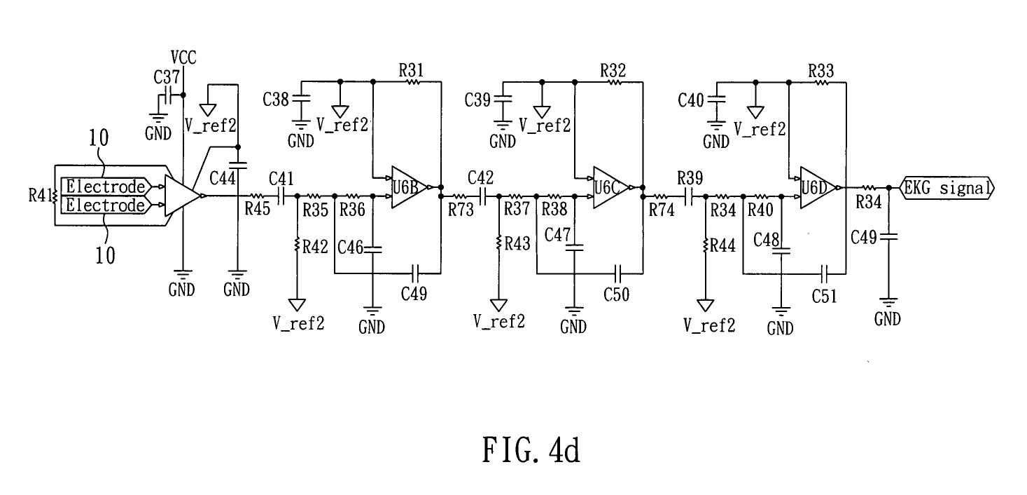

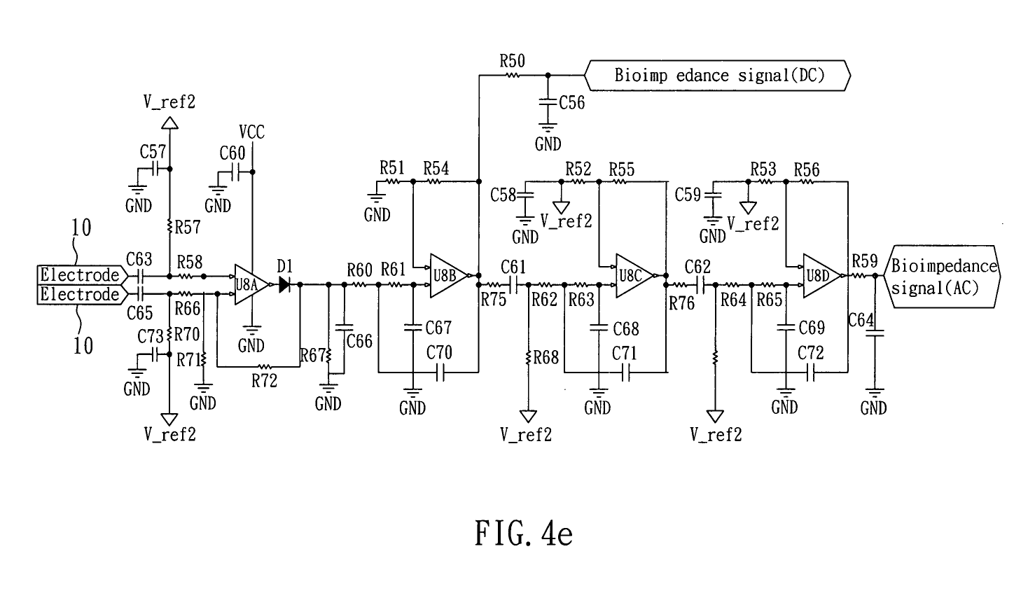

Please refer to FIG. 3. FIG. 3 is a circuit diagram of the portable multi-parameter physiological monitoring and recording device in accordance with one preferred embodiment of the present invention, wherein the measured physiological parameter signals 1-8 are passed through the multiplexer 12 and the controller 13, and then audio signals are outputted from the connection interface 16. Please refer to FIG. 4a to FIG. 4e. FIG. 4a-4e are circuit diagrams of the detection channel units of the portable multi-parameter physiological monitoring and recording device in accordance with one preferred embodiment of the present invention, wherein the detection channel units are used for measuring room temperature signals, body temperature signals, XYZ three-axis acceleration signals, EKG signals and breathing signals respectively.

In view of the foregoing, it is known that the portable multi-parameter physiological monitoring and recording device according to the present invention synthesizes the measured results as a single audio signal and records the same as an audio file, and hence the present invention, which is convenient and easy to use, achieves high portability. Since audio signals are easily recorded and transferred for general users, common recording devices can be used for storing the measured results and an audio signal can be transferred through Bluetooth wireless transmission, the present invention can be widely applied in the domestic devices, remote care device and sport equipments. Therefore, the present invention is very convenient for the elderly who are not good to use computer devices or for those who do not have computer devices, thereby achieving a high efficiency in usage.

Although the present invention has been explained in relation to its preferred embodiment, it is to be understood that many other possible modifications and variations can be made without departing from the scope of the invention as hereinafter claimed.

What is claimed is:

1. A portable multi-parameter physiological monitoring and recording device, which is used for measuring multiple physiological signals of a person. These measured results can be outputted as an audio signal and saved as an audio file, comprising:
Several sensors attached on specified locations on the body skin of a person for sensing the corresponding physiological signals respectively;
multiple-detection channel units respectively connected to the corresponding sensors to amplify the received physiological signals;
a multiplexer connected to the detection channel units for capturing the amplified physiological parameter signals;
a clock generator for generating clock signal in the audio-frequency range;
a controller connected to the multiplexer and the clock generator for controlling the multiplexer to capture the amplified physiological signals based on the clock signals generated by the clock generator;
an analog switch connected to the detection channel units and the multiplexer for outputting the amplified physiological signals as an audio signal;
a recording unit connected to the analog switch and the controller for recording the audio signal as an audio file under control of the controller; and
a storage unit connected to the recording unit for storing the audio file.

2. The portable multi-parameter physiological monitoring and recording device as claimed in claim 1, further comprising a connection interface connecting to the analog switch for outputting the audio signals.

3. The portable multi-parameter physiological monitoring and recording device as claimed in claim 2, wherein the connection interface is connected to an external storage device through cable or wireless transmission for storing the audio signal outputted from the connection interface as the audio file.

4. The portable multi-parameter physiological monitoring and recording device as claimed in claim 3, wherein the external storage device is provided to process the received audio signal to proceed analysis or determination instantly.

5. The portable multi-parameter physiological monitoring and recording device as claimed in claim 3, wherein the external storage device is a computer device, a recording device or a personal digital assistant.

6. The portable multi-parameter physiological monitoring and recording device as claimed in claim 3, wherein the external storage device is connected to the connection interface to transfer the audio signals through audio cables.

7. The portable multi-parameter physiological monitoring and recording device as claimed in claim 3, wherein the external storage device is connected to the connection interface to transfer the audio signals through Bluetooth wireless transmission.

8. The portable multi-parameter physiological monitoring and recording device as claimed in claim 1, further comprising a display panel connected to the controller for displaying the amplified physiological signals captured by the multiplexer.

9. The portable multi-parameter physiological monitoring and recording device as claimed in claim 1, wherein each detection channel unit includes an isolation layer for separating the skin of the person and the circuits of the physiological monitoring and recording device to prevent leakage current or noise harming the human body.

10. The portable multi-parameter physiological monitoring and recording device as claimed in claim 1, wherein the analog switch is provided with a first reference voltage value, a second reference voltage value and a third reference voltage value for constituting a start signal, an interval signal and a terminate signal, and the amplified physiological parameter signals outputted from the analog switch are produced by outputting the start signal first, then sequentially outputting the captured amplified physiological parameter signals with outputting the interval signal in between different physiological parameter signals, and finally outputting the terminate signal.

11. The portable multi-parameter physiological monitoring and recording device as claimed in claim 1, wherein the physiological signals are Electrocardiography (EKG) signals, breathing signals, body impedance signals, room temperature signals, body temperature signals or XYZ three-axis acceleration signals.

12. The portable multi-parameter physiological monitoring and recording device as claimed in claim 1, wherein the frequency of the audio signal is between 200 Hz and 20 KHz.
